(19) United States
(12) Patent Application Publication
(10) Pub. No.: US 2024/0122460 A1
(43) Pub. Date: Apr. 18, 2024

(54) FLEXIBLE ELECTRONIC CIRCUIT BOARD FOR A MULTI-CAMERA ENDOSCOPE

(71) Applicant: EndoChoice, Inc., Alpharetta, GA (US)

(72) Inventors: Yaniv Kirma, Karcur (IL); Moshe LEVI, Ganey Tikva (IL); Amram Aizenfeld, Ramot Menashe (IL)

(73) Assignee: EndoChoice, Inc., Alpharetta, GA (US)

(21) Appl. No.: 18/395,165

(22) Filed: Dec. 22, 2023

Related U.S. Application Data
(63) Continuation of application No. 17/968,903, filed on Oct. 19, 2022, now Pat. No. 11,889,986.
Application No. 17/968,903 is a continuation of application No. 17/122,674, filed on Dec. 15, 2020, now Pat. No. 11,497,388.
Application No. 17/122,674 is a continuation of application No. 15/730,202, filed on Oct. 11, 2017, now Pat. No. 10,898,063.
Application No. 15/730,202 is a continuation of application No. 13/992,014, filed on Jun. 6, 2013, now Pat. No. 9,814,374.
(60) Provisional application No. 61/421,238, filed on Dec. 9, 2010.

(51) Int. Cl. A61B 1/04; A61B 1/00; A61B 1/05; H04N 23/45; H04N 23/54; H04N 23/56

(52) U.S. Cl. CPC A61B 1/042; A61B 1/00091; A61B 1/00094; A61B 1/00096; A61B 1/00177; A61B 1/05; H04N 23/45; H04N 23/54; H04N 23/56; H04N 23/555

(57) ABSTRACT

There is provided herein a flexible electronic circuit board for a tip section of a multi-camera endoscope, the circuit board comprising a front camera surface configured to carry a forward looking camera, a first side camera surface configured to carry a first side looking camera, a second side camera surface configured to carry a second side looking camera, one or more front illuminator surfaces a configured to carry one or more front illuminators to essentially illuminate the FOV of the forward looking camera, one or more side illuminator surfaces configured to carry one or more side illuminators to essentially illuminate the FOV of the first side looking camera and one or more side illuminator surfaces configured to carry one or more side illuminators to essentially illuminate the FOV of the second side looking camera.

### CROSS REFERENCE

The present application is a national stage application of PCT/IL2011/050049, which was filed on Dec. 8, 2011, and relies on U.S. Provisional Patent Application No. 61/421,238, filed on Dec. 9, 2010 for priority. All of the aforementioned applications are herein incorporated by reference.

### FIELD

Embodiments of the disclosure relate to a multi-camera endoscope having a flexible electronic circuit board.

### Background

Endoscopes have attained great acceptance within the medical community, since they provide a means for performing procedures with minimal patient trauma, while enabling the physician to view the internal anatomy of the patient. Over the years, numerous endoscopes have been developed and categorized according to specific applications, such as cystoscopy, colonoscopy, laparoscopy, upper GI endoscopy and others. Endo scopes may be inserted into the body's natural orifices or through an incision in the skin.

An endoscope is usually an elongated tubular shaft, rigid or flexible, having a video camera or a fiber optic lens assembly at its distal end. The shaft is connected to a handle, which sometimes includes an ocular for direct viewing. Viewing is also usually possible via an external screen. Various surgical tools may be inserted through a working channel in the endoscope for performing different surgical procedures.

Endoscopes, such as colonoscopes, that are currently being used, typically have a front camera for viewing the internal organ, such as the colon, an illuminator, a fluid injector for cleaning the camera lens and sometimes also the illuminator and a working channel for insertion of surgical tools, for example, for removing polyps found in the colon. Often, endoscopes also have fluid injectors (“jet”) for cleaning a body cavity, such as the colon, into which they are inserted. The illuminators commonly used are fiber optics which transmit light, generated remotely, to the endoscope tip section. The use of light-emitting diodes (LEDs) for illumination is also known.

Among the disadvantages of such endoscopes, are their limited field of view and their complicated packing of all the required elements, such as electronics and fiber optics together with fluid carrying elements in the small sized endoscope tip section.

There is thus a need in the art for endoscopes, such as colonoscopies, that allow a broader field of view and also enable the efficient packing of all necessary elements in the tip section, while maintaining their function.

The foregoing examples of the related art and limitations related therewith are intended to be illustrative and not exclusive. Other limitations of the related art will become apparent to those of skill in the art upon a reading of the specification and a study of the figures.

### Summary

The following embodiments and aspects thereof are described and illustrated in conjunction with systems, tools and methods which are meant to be exemplary and illustrative, not limiting in scope.

According to some embodiments, there is provided a flexible electronic circuit board for a tip section of a multi-camera endoscope, the circuit board comprising: a front camera surface configured to carry a forward looking camera; a first side camera surface configured to carry a first side looking camera; a second side camera surface configured to carry a second side looking camera; one or more front illuminator surfaces configured to carry one or more front illuminators to essentially illuminate the Field Of View (FOV) of the forward looking camera; one or more side illuminator surfaces configured to carry one or more side illuminators to essentially illuminate the FOV of the first side looking camera; and one or more side illuminator surfaces configured to carry one or more side illuminators to essentially illuminate the FOV of the second side looking camera. The term “essentially illuminate the FOV of” may also refer to illuminating only a part of the FOV. The one or more front illuminator surfaces may include three front illuminator surfaces.

The front camera surface and said one or more front illuminator surfaces may be essentially parallel to each other, and essentially perpendicular to a center portion of said flexible electronic circuit board, when said flexible electronic circuit board is in a folded configuration.

According to some embodiments, when said flexible electronic circuit board is in a folded configuration, said first side camera surface and said second side camera surface are essentially parallel to each other, such that said first side looking camera and said second side looking camera are directed to opposing sides.

According to some embodiments, when said flexible electronic circuit board is in a folded configuration, said first side camera surface and said second side camera surface are essentially perpendicular to a center portion of said flexible electronic circuit board.

According to some embodiments, when said flexible electronic circuit board is in a folded configuration, said first side camera surface and said second side camera surface are essentially perpendicular to said front camera surface.

According to some embodiments, said one or more side illuminator surfaces comprises two side illuminator surfaces.

According to some embodiments, said two side illuminator surfaces are configured to carry two side illuminators to essentially illuminate the FOV of the first side looking camera, and wherein, when said flexible electronic circuit board is in a folded configuration, said two side illuminator surfaces are essentially parallel to each other and essentially perpendicular to said first side camera surface, which is located between them.

According to some embodiments, said two side illuminator surfaces are configured to carry two side illuminators to essentially illuminate the FOV of the second side looking camera, and wherein, when said flexible electronic circuit board is in a folded configuration, said two side illuminator surfaces are essentially parallel to each other and essentially perpendicular to said second side camera surface, which is located between them.

According to some embodiments, there is provided a tip section of a multi-camera endoscope, the tip section comprising: a folded flexible electronic circuit board for a tip section of a multi-camera endoscope, the circuit board comprising: a front camera surface configured to carry a forward looking camera; a first side camera surface configured to carry a first side looking camera; a second side camera surface configured to carry a second side looking camera; one or more front illuminator surfaces a configured to carry one or more front illuminators to essentially illuminate the FOV of the forward looking camera; one or more side illuminator surfaces configured to carry one or more side illuminators to essentially illuminate the FOV of the first side looking camera; and one or more side illuminator surfaces configured to carry one or more side illuminators to essentially illuminate the FOV of the second side looking camera; and a flexible electronic circuit board holder configured to retain said flexible electronic circuit board in a folded position.

According to some embodiments, the tip section of an endoscope (such as a colonoscope) is the most distal part of the endoscope which terminates the endoscope. The tip section is turnable by way of a bending section connected thereto.

According to some embodiments, the tip section further includes a fluid channeling component adapted to channel fluid for insufflations and/or irrigation. The fluid channeling component may be a unitary component comprising a front fluid channel leading to a front opening at a distal end of said unitary fluid channeling component, for cleaning one or more front optical elements of said tip section, and a side fluid channel leading to a left side opening and to a right side opening in said unitary fluid channeling component, for cleaning side optical elements of said tip section. The unitary fluid channeling component may further include a working channel adapted for the insertion of a medical tool. The unitary fluid channeling component may further include a jet fluid channel adapted to clean a body cavity into which said endoscope is inserted.

According to some embodiments, the one or more front illuminator surfaces comprises three front illuminator surfaces.

According to some embodiments, said front camera surface and said one or more front illuminator surfaces are essentially parallel to each other, and essentially perpendicular to a center portion of said flexible electronic circuit board.

According to some embodiments, said first side camera surface and said second side camera surface are essentially parallel to each other, such that said first side looking camera and said second side looking camera are directed to opposing sides. According to some embodiments, said first side camera surface and said second side camera surface are essentially perpendicular to a center portion of said flexible electronic circuit board. According to some embodiments, said first side camera surface and said second side camera surface are essentially perpendicular to said front camera surface.

According to some embodiments, said one or more side illuminator surfaces comprises two side illuminator surfaces. According to some embodiments, said two side illuminator surfaces are configured to carry two side illuminators to essentially illuminate the FOV of the first side looking camera, and wherein, when said flexible electronic circuit board is in a folded configuration, said two side illuminator surfaces are essentially parallel to each other and essentially perpendicular to said first side camera surface, which is located between them.

According to some embodiments, said two side illuminator surfaces are configured to carry two side illuminators to essentially illuminate the FOV of the second side looking camera, and wherein, when said flexible electronic circuit board is in a folded configuration, said two side illuminator surfaces are essentially parallel to each other and essentially perpendicular to said second side camera surface, which is located between them.

According to some embodiments, the flexible electronic circuit board holder may be configured to be used as a heat sink for one or more of the side and front illuminators.

According to some embodiments, the tip section has having a diameter of about 17 mm or less. According to some embodiments, the tip section has having a diameter of about 12 mm or less. According to some embodiments, the tip section has having a diameter of about 10 mm or less.

According to some embodiments, there is provided herein a multi-camera endoscope, such as a colonoscope, comprising the tip section disclosed herein. According to some embodiments, the tip section of an endoscope (such a colonoscope) is the most distal part of the endoscope which terminates the endoscope. The tip section is turnable by way of a bending section connected thereto.

More details and features of the current invention and its embodiments may be found in the description and the attached drawings.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. It is intended that the embodiments and figures disclosed herein are to be considered illustrative rather than restrictive. The figures are listed below:

FIG. 1 schematically depicts an external isometric view of a tip section of an endoscope having multiple fields of view, according to an exemplary embodiment of the current invention;

FIG. 2 schematically depicts an isometric view of a folded flexible electronic circuit board carrying a front view camera, two side view cameras and illumination sources, according to an exemplary embodiment of the current invention;

FIG. 3 schematically depicts an isometric view of a folded flexible electronic circuit board, according to an exemplary embodiment of the current invention;

FIG. 4 schematically depicts an isometric view of a flexible electronic circuit board in an unfolded (flat) configuration, according to an exemplary embodiment of the current invention;

FIG. 5 schematically depicts an isometric exploded view of a folded flexible electronic circuit board carrying cameras and illumination sources and a flexible electronic circuit board holder, according to an exemplary embodiment of the current invention;

FIG. 6 schematically depicts an isometric view of a folded flexible electronic circuit board carrying cameras and illumination sources and a flexible electronic circuit board holder, according to an exemplary embodiment of the current invention;

FIG. 7 schematically depicts an isometric view of a folded flexible electronic circuit board carrying cameras and illumination sources, a flexible electronic circuit board holder, and a fluid channeling component, according to an exemplary embodiment of the current invention; and,

FIG. 8 schematically depicts an isometric view of a folded flexible electronic circuit board carrying cameras and illumination sources, a flexible electronic circuit board holder, a fluid channeling component, and a tip cover (in an exploded view), according to an exemplary embodiment of the current invention.

### DETAILED DESCRIPTION

While a number of exemplary aspects and embodiments have been discussed above, those of skill in the art will recognize certain modifications, permutations, additions and sub-combinations thereof. It is therefore intended that the following appended claims and claims hereafter introduced be interpreted to include all such modifications, permutations, additions and sub-combinations as are within their true spirit and scope.

In the description and claims of the application, each of the words “comprise” “include” and “have”, and forms thereof, are not necessarily limited to members in a list with which the words may be associated.

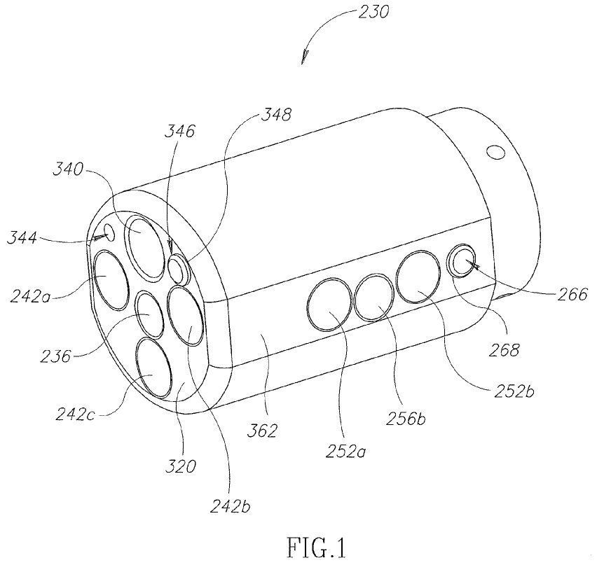

FIG. 1 schematically depicts an external isometric view of a tip section of an endoscope having multiple fields of view according to an exemplary embodiment of the current invention.

According to an exemplary embodiment of the current invention, tip section 230 of an endoscope which comprises at least a forwards looking camera and at least one side looking camera. Tip section 230 is turnable by way of flexible shaft (not shown) which may also be referred to as a bending section, for example a vertebra mechanism).

In some embodiments, the front-looking camera and/or any of the side-looking cameras comprises a Charge Coupled Device (CCD) or a Complementary Metal Oxide Semiconductor (CMOS) image sensor.

It is noted that the term “endoscope” as mentioned to herein may refer particularly to a colonoscope, according to some embodiments, but is not limited only to colonoscopes. The term “endoscope” may refer to any instrument used to examine the interior of a hollow organ or cavity of the body.

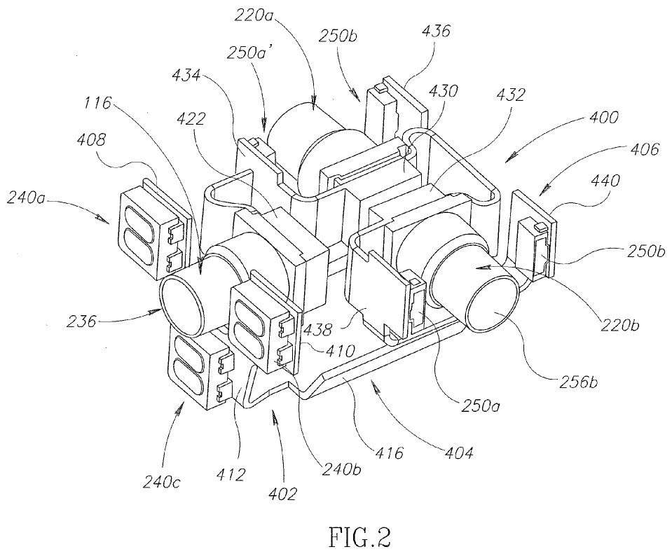

Tip section 230 includes front optical assembly 236 of forwards looking camera 116 (seen for example in FIGS. 2 and 5-8) on the front face 320 of tip section 230. Optical axis of forwards looking camera 116 is substantially directed along the long dimension of the endoscope. However, since forward looking camera 116 is typically a wide angle camera, its Field Of View (FOV) may include viewing directions at large angles to its optical axis. Additionally, optical windows 242a, 242b and 242c of LEDs 240a, 240b and 240c, respectively, (seen for example in FIGS. 2 and 5-8) are also located on front face 320 of tip section 230. It should be noted that number of illumination sources such as LEDs used for illumination of the FOV may vary (for example, 1-5 LEDs may be used on front face 320 of tip section 230). Distal opening 340 of a working channel (not shown) is also located on front face 320 of tip section 230, such that a surgical tool inserted through working channel tube, and through the working channel in the endoscope's tip section 230 and deployed beyond front face 320 may be viewed by forwards looking camera 116.

Distal opening 344 of a jet fluid channel is also located on front face 320 of tip section 230. Distal opening 344 of a jet fluid channel may be used for providing high pressure jet of fluid such as water or saline for cleaning the walls of the body cavity.

Also located on front face 320 of tip section 230 is an irrigation and insufflation (I/I) injector 346 having a nozzle 348 aimed at front optical assembly 236. I/I injector 346 may be used for injecting fluid (liquid and/or gas) to wash contaminants such as blood, feces and other debris from front optical assembly 236 of forwards looking camera. Optionally the same injector is used for cleaning front optical assembly 236 and one two or all of optical windows 242a, 242b and 242c. I/I injector 346 may be fed by fluid such as water and/or gas which may be used for cleaning and/or inflating a body cavity.

Visible on the side wall 362 of tip section 230 is the side camera (side looking camera) element 256b of side looking camera 220b and optical windows 252a and 252b of LEDs 250a and 250b for camera 220b. A second side looking camera, 220a, is not shown in FIG. 1 but can be seen for example in FIGS. 2 and 5-6, along with its optical assemblies 256a and optical windows 252a′ and 252b′ of LEDs 250a′ and 250b′ of camera 220a. Optical axis of side looking camera 220a is substantially directed perpendicular to the long dimension of the endoscope. Optical axis of side looking camera 220b is substantially directed perpendicular to the long dimension of the endoscope. However, since side looking cameras 220a and 220b is typically a wide angle camera, its field of view may include viewing directions at large angles to its optical axis.

I/I injector 266 having a nozzle 268 aimed at side optical assembly 256b may be used for injecting fluid to wash contaminants such as blood, feces and other debris from side optical assembly 256b of side looking camera. The fluid may include gas which may be used for inflating a body cavity. Optionally the same injector is used for cleaning both side optical assembly 256b and optical windows 252a and/or 252b. It is noted that according to some embodiments, the tip may include more than one window and LEDs, on the side and more than one window and LEDs in the front (for example, 1-5 windows and two LEDs on the side). Similar configuration of I/I injector and nozzle exists for cleaning optical assembly 256a and optical windows 252a′ and 252b′ located on the other side of tip 230. The I/I injectors are configured to clean all or a part of these windows/LEDs. I/I injectors 346 and 266 may be fed from same channel.

It is noted that the side wall 362 has a form of an essentially flat surface which assists in directing the cleaning fluid injected from I/I injector 266 towards side optical assembly 256b and optical windows 252a and/or 252b. Lack of such flat surface may result in dripping of the cleaning fluid along the curved surface of tip section 230 of the endoscope without performing the desired cleaning action.

It should be noted that while only one side looking camera is seen in FIG. 1, preferably at least two side looking cameras may be located within tip section 230. When two side looking cameras are used, the side looking cameras are preferably installed such that their field of views are substantially opposing. However, different configurations and number of side looking cameras are possible within the general scope of the current invention.

A significant problem always existed in the art when attempts were made to pack all necessary components into the small inner volume of the endoscope. This problem dramatically increases when three cameras and respective illumination sources (such as LEDs) should be packed in the tip of the endoscope, as disclosed herein in accordance to some embodiments of the present invention. There is thus provided, according to some embodiments of the invention, a flexible electronic circuit for carrying and packing within the limited inner volume of the endoscope's tip, at least a front camera and one or more (for example two) side view cameras and their respective illumination sources.

According to some embodiments, the flexible circuit board consumes less space and leaves more volume for additional necessary features. The flexibility of the board adds another dimension in space that can be used for components positioning.

The use of the circuit board according to embodiments of the invention can significantly increase reliability of the electric modules connection thereto as no wires are for components connectivity. In addition, according to some embodiments, the components assembly can be machined and automatic.

The use of the circuit board according to embodiments of the invention, may also allow components (parts) movement and maneuverability during assembly of the camera head (tip of the endoscope) while maintaining high level of reliability. The use of the circuit board according to embodiments of the invention, may also simplify the (tip) assembling process.

According to some embodiments, the flexible circuit board is connected to the control unit via multi wire cable; this cable is welded on the board in a designated location freeing additional space within the tip assembly and adding flexibility to cable access. Assembling the multi wire cable directly to the electrical components was a major challenge which is mitigated by the use of the flexible board according to embodiments of the invention.

FIG. 2 schematically depicts an isometric view of a folded flexible electronic circuit board carrying a front view camera, two side view cameras and illumination sources, according to embodiments of the invention.

Flexible electronic circuit board 400, shown here in a folded configuration, is configured to carry forward looking camera 116; LEDs 240a, 240b and 240c positioned to essentially illuminate the Field Of View (FOV) of forward looking camera 116; side looking cameras 220b; LEDs 250a and 250b positioned to essentially illuminate the Field Of View (FOV) of side looking cameras 220b; side looking cameras 220a and LEDs 250a′ and 250b′ positioned to essentially illuminate the Field Of View (FOV) of side looking cameras 220a.

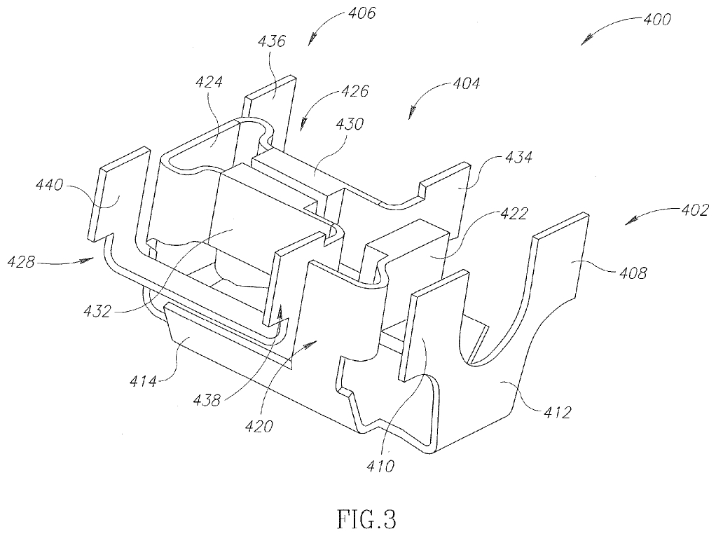

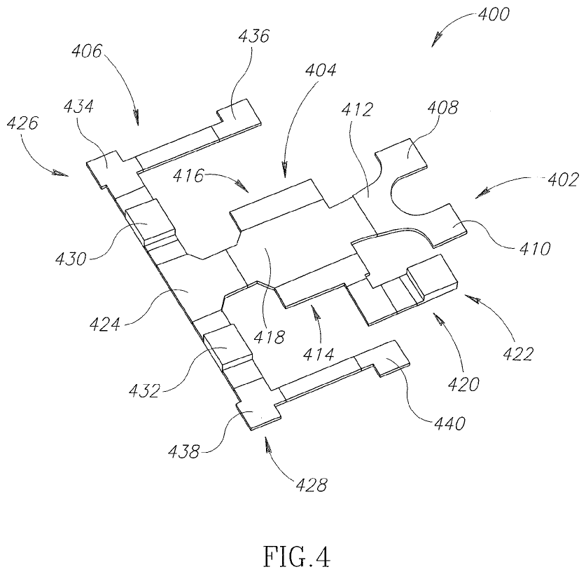

As can also be seen in FIGS. 3 and 4, which schematically depict isometric views of flat and folded flexible electronic circuit board, respectively, according to embodiments of the invention, flexible electronic circuit board 400 includes three sections: front section 402, main section 404 and rear section 406.

Front section 402 of flexible electronic circuit board 400 includes first front LED surface 408, second front LED surface 410 and a bottom front LED surface 412. First front LED surface 408, second front LED surface 410 and a bottom front LED surface 412 are flat surfaces formed from a unitary piece of a PCB layer. First front LED surface 408 is adapted to carry front LED 240a, second front LED surface 410 is adapted to carry front LED 240b and a bottom front LED surface 412 is adapted to carry front LED 240c. First front LED surface 408, second front LED surface 410 and a bottom front LED surface 412 form an arc shape between them which is configured to support forward looking camera 116.

Front section 402 of flexible electronic circuit board 400 is connected to main section 404 through bottom section 412. Main section 404 of flexible electronic circuit board 400 includes a center portion 418, a first foldable side panel 414 and a second foldable side panel 416. When flexible electronic circuit board 400 is in a folded configuration, first foldable side panel 414 and a second foldable side panel 416 are configured to fold upwards (towards the length axis of the endoscope tip), for example, as shown herein, forming an angle of about 45 degrees with center portion 418 of main section 404. First foldable side panel 414 also includes an arm section 420, extending therefrom, having a front sensor surface 422 (may also be referred to as a camera surface) adapted to carry forward looking camera 116. When flexible electronic circuit board 400 is in folded position, arm section 420 is folded to be essentially perpendicular to center portion 418 of main section 404, and front sensor surface 422 is folded to be essentially perpendicular to center portion 418 and to arm section 420, such that it faces forwards, essentially at the same direction of first front LED surface 408, second front LED surface 410 and a bottom front LED surface 412. This configuration enables forward looking camera 116 and LEDs 240a-c to face the same direction.

As described hereinabove, main section 404 is connected to bottom section 412 of front section 402. On the opposing end of main section 404, it is connected to rear section 406.

Rear section 406 includes a rear central portion 424. Rear central portion 424 is connected to a first rear arm section 426, extending from one side of rear central portion 424 and to a second rear arm section 428, extending from the opposing side of rear central portion 424.

First rear arm section 426 includes a first side sensor surface 430 (adapted to carry side looking camera 220a). Second rear arm section 428 includes a second side sensor surface 432 (adapted to carry side looking camera 220b).

First rear arm section 426 further includes a first side LED surface 434 and a second side LED surface 436, adapted to carry side LEDs 250a′ and 250b′, respectively. Second rear arm section 428 further includes a third side LED surface 438 and a fourth side LED surface 440, adapted to carry side LEDs 250a and 250b, respectively.

According to some embodiments, front sensor surface 422 (which is adapted carry forward looking camera 116), first side sensor surface 430 and second side sensor surface 432 (which are adapted carry side looking cameras 220a and 220b) are thicker than the front and side LED surfaces. For example, the sensor surface thickness is configured for locating the sensor (of the camera) such that the welding pins of the sensor wrap the surface and are welded on the opposite side of the sensor in specific welding pads.

The sensor surfaces may be rigid and used as basis for the camera assembly. The height of the sensor surface has significant importance allowing the sensor conductors to bend in a way they will directly reach the welding pads on the opposite side of the sensor rigid surface. The rigid basis also serves as electrical ground filtering electromagnetic noise to and from the sensor and thus increasing signal integrity.

When flexible electronic circuit board 400 is in a folded configuration, rear central portion 424 is folded upwards, perpendicularly to center portion 418 of main section 404. First side sensor surface 430 and second side sensor surface 432 are positioned perpendicularly to center portion 418 and also perpendicularly rear central portion 424. In addition, first side sensor surface 430 and second side sensor surface 432 are positioned essentially parallel and “back to back” to each other such that when they carry side looking camera 220a and side looking camera 220b, these cameras view opposing sides. First side LED surface 434 and a second side LED surface 436 are positioned perpendicularly to first side sensor surface 430 and adapted to carry, on their inner sides, side LEDs 250a′ and 250b′, respectively, such that LEDs 250a′ and 250b′ are positioned in proximity to side looking camera 220a. Third side LED surface 438 and a fourth side LED surface 440 are positioned perpendicularly to second side sensor surface 432 and adapted to carry, on their inner sides, side LEDs 250a and 250b, respectively, such that LEDs 250a and 250b are positioned in proximity to side looking camera 220b.

According to some embodiments of the invention, front section 402, main section 404 and rear section 406 of flexible electronic circuit board 400 are all integrally formed from a unitary piece of circuit board layer.

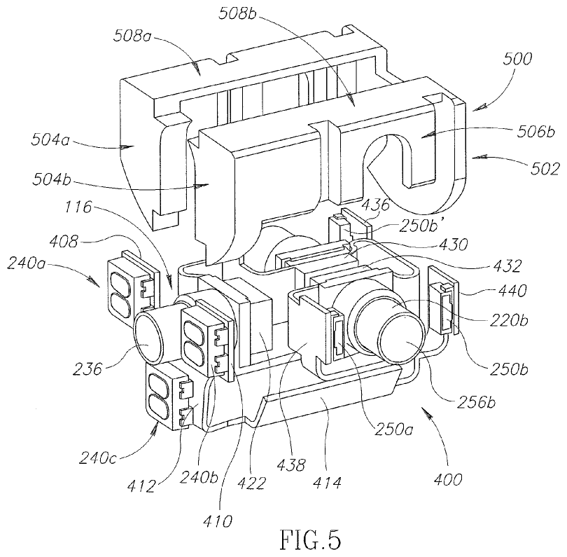

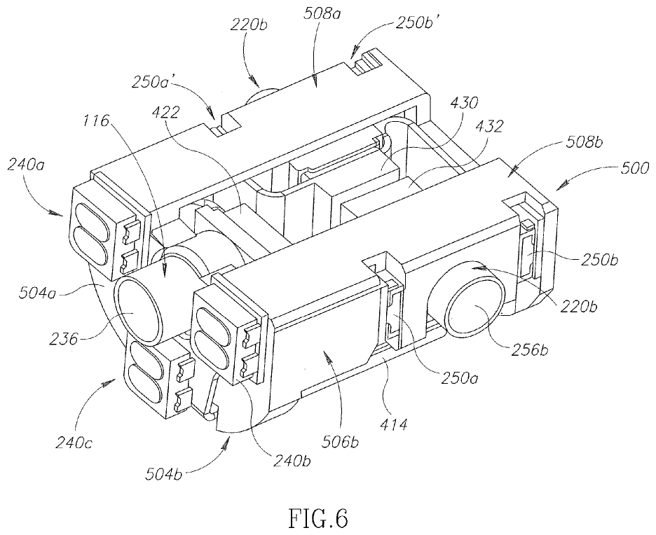

Reference is now made to FIGS. 5 and 6 which schematically depict isometric views (FIG. 5 shows an exploded view) of a folded flexible electronic circuit board carrying cameras and illumination sources and a flexible electronic circuit board holder, according to an exemplary embodiment of the current invention.

Similar to FIG. 2, flexible electronic circuit board 400, shown in FIG. 5 in its folded configuration, is configured to carry forward looking camera 116; LEDs 240a, 240b and 240c positioned to illuminate essentially the Field Of View (FOV) of forward looking camera 116; side looking cameras 220b; LEDs 250a and 250b positioned to illuminate essentially the Field Of View (FOV) of side looking cameras 220b; side looking cameras 220a and LEDs 250a′ and 250b′ positioned to illuminate essentially the Field Of View (FOV) of side looking cameras 220a.

Flexible electronic circuit board holder 500 is adapted to hold flexible electronic circuit board 400 in its desired folded position, and secure the front and side looking cameras and their corresponding illuminators in place. As shown in FIG. 5, flexible electronic circuit board holder 500 is a unitary piece of rigid material, such as brass, stainless steel, aluminum or any other material.

According to some embodiments, the use of metal for the construction of the flexible electronic circuit board holder is important for electric conductivity and heat transfer purposes. The flexible electronic circuit board holder, according to embodiments of the invention, (such as flexible electronic circuit board holder 500) can be used as a heat sink for some or all of the electronic components located at the tip section, particularly illuminators (such as side or front LEDs) and reduce overall temperature of the endoscope tip. This may solve or at least mitigate a major problem of raised temperatures of endoscope tip and/or any of its components, particularly when using LED illuminators.

Flexible electronic circuit board holder 500 includes a back portion 502 adapted to support second side LED surface 436 and fourth side LED surface 440.

Flexible electronic circuit board holder 500 further includes front portions 504a and 504b, supporting the back sides (opposing to the sides where the LEDs are attached) of first front LED surface 408 and second front LED surface 410, respectively.

Flexible electronic circuit board holder 500 further includes two side portions 506a (not shown) and 506b on the two opposing sides of flexible electronic circuit board holder 500. Each of side portions 506a and 506b include two small openings for the side LEDs (250a, 250b, 250a′, 250b′) and one opening for side looking camera 220b and 220a (not shown). Side portions 506a and 506b of flexible electronic circuit board holder 500 abut first and second side foldable panels 416 and 414, respectively, of flexible electronic circuit board 400.

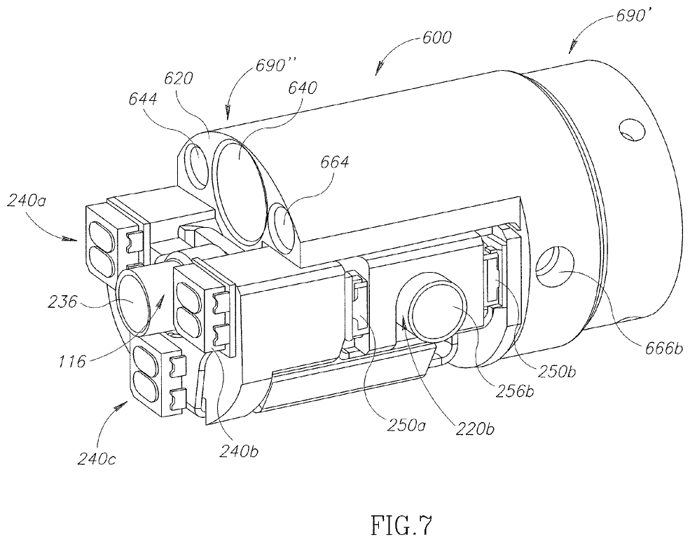

Flexible electronic circuit board holder 500 further includes a top part including top portions 508a and 508b (the top part of the flexible electronic circuit board holder may also include one top portion) covering the top part of flexible electronic circuit board 400 and configured to support fluid channeling component 600 (FIG. 7).

Reference is now made to FIG. 7, which schematically depicts an isometric view of a folded flexible electronic circuit board carrying cameras and illumination sources, a flexible electronic circuit board holder, and a fluid channeling component, according to an exemplary embodiment of the current invention. FIG. 6 schematically depicts an isometric view of a folded flexible electronic circuit board carrying cameras and illumination sources and a flexible electronic circuit board holder. FIG. 7 adds to the configuration of FIG. 6, a fluid channeling component 600, which includes irrigation and insufflation (UI) channels, jet channel and a working channel. Fluid channeling component 600 is a separate component from flexible electronic circuit board 400. This configuration is adapted to separate the fluid channels and working channel, which are located in fluid channeling component 600 from the sensitive electronic and optical parts which are located in the area of flexible electronic circuit board 400.

Fluid channeling component 600 (or according to some embodiments, a unitary fluid channeling component), according to some embodiments, may generally include two parts: a proximal fluid channeling component section 690′ and a distal fluid channeling component section 690″. Proximal fluid channeling component section 690′ may have an essentially cylindrical shape. Distal unitary channeling component section 690″ may partially continue the cylindrical shape of proximal fluid channeling component section 690′ and may have a shape of a partial cylinder (optionally elongated partial cylinder), having only a fraction of the cylinder (along the height axis of the cylinder), wherein another fraction of the cylinder (along the height axis of the cylinder) is missing. Distal fluid channeling component section 690″ may be integrally formed as a unitary block with proximal fluid channeling component section 690′. The height of distal fluid channeling component section 690″ may by higher than that of proximal fluid channeling component section 690′. In the case of distal fluid channeling component section 690″, the shape of the partial cylinder (for example, partial cylinder having only a fraction of a cylinder shape along one side of the height axis) may provide a space to accommodate flexible electronic circuit board 400 and flexible electronic circuit board holder 500.

Front face 620 of distal fluid channeling component section 690″ includes a distal opening 640 of working channel (located inside fluid channeling component 690, not shown). Front face 620 of distal fluid channeling component section 690″ further includes distal opening 644 of a jet fluid channel which may be used for providing high pressure jet of fluid such as water or saline for cleaning the walls of the body cavity (such as the colon) and optionally for suction. Front face 620 of distal fluid channeling component section 690″ further includes irrigation and insufflation (I/I) opening 664 which may be used for injecting fluid (liquid and/or gas) to wash contaminants such as blood, feces and other debris from front optical assembly 236 of forwards looking camera 116.

Proximal fluid channeling component section 690′ of fluid channeling component 600 includes I/I openings 666a (not shown) and 666b aimed at side optical assembly 256a and 256b, respectively, and used for injecting fluid (the term “fluid” may also include gas and/or liquid) to wash contaminants such as blood, feces and other debris from side optical assemblies 256a and 256b of side looking cameras 220a and 220b. According to some embodiments, the injectors may supply liquid for cleaning any of the tip elements (such as any optical assembly, windows, LEDs, and other elements).

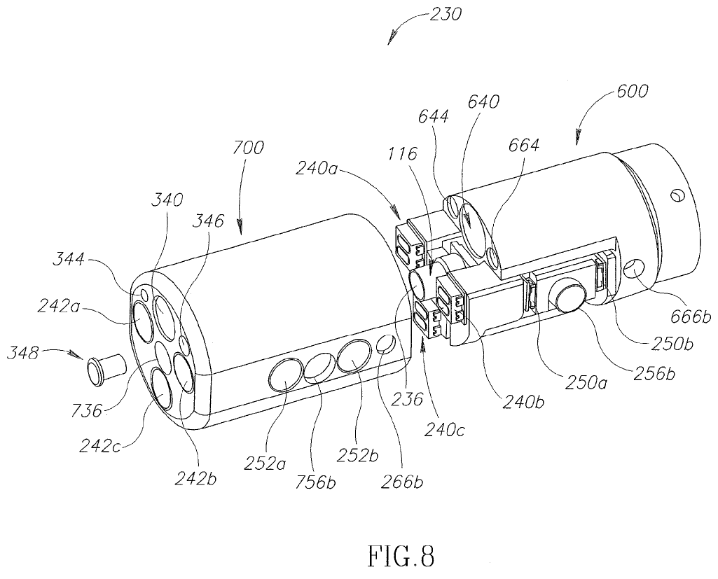

Reference is now made to FIG. 8, which schematically depicts an isometric view of a folded flexible electronic circuit board carrying cameras and illumination sources, a flexible electronic circuit board holder, a fluid channeling component, and a tip cover (in an exploded view), which together form a tip section of an endoscope, according to an exemplary embodiment of the current invention.

Fluid channeling component 600, flexible electronic circuit board 400 and flexible electronic circuit board holder 500 are described in FIGS. 6 and 7. Tip cover 700 is designed to fit over the inner parts of the tip section 230, and to provide protection to the internal components in the inner part.

Tip cover 700 includes hole 736 configured to align with front optical assembly 236 of forwards looking camera 116; optical windows 242a, 242b and 242c of LEDs 240a, 240b and 240c (seen for example in FIGS. 2 and 5-8); distal opening 340 of a working channel (not shown); distal opening 344 of a jet fluid channel; I/I injector 346 having a nozzle 348 (aligning with opening 664 of Fluid channeling component 600); holes 756a (not shown) and 756a configured to align with side optical assemblies 256a and 256b of side looking cameras 220a and 220b; optical windows 252a and 252b of LEDs 250a and 250b for camera 220a; and optical windows 252a′ and 252b′ of LEDs 250a′ and 250b′ for camera 220b; side holes 266a (not shown) and 266b adapted to align with I/I openings 666a (not shown) and 666b.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1-20. (canceled)

21. An electronic assembly for a medical device, wherein the electronic assembly comprises:
a first optical assembly;
a second optical assembly;
a flexible circuit board having a bent configuration and an unbent configuration, and wherein the flexible circuit board comprises: a distal section configured to hold the first optical assembly and a proximal section configured to hold the second optical assembly; and
a holder configured to hold the flexible circuit board and including a first space configured to receive the first optical assembly and a second space configured to receive the second optical assembly.

22. The electronic assembly of claim 21, wherein the distal section of the flexible circuit board includes a first distal portion, a second distal portion, and a proximal portion that together form an arc shape configured to support the first optical assembly.

23. The electronic assembly of claim 21, wherein the holder comprises:
a first planar distal surface extending transverse to a central longitudinal axis of the first optical assembly; and
a second planar distal surface extending transverse to the central longitudinal axis of the first optical assembly;
wherein the space of the holder extends between the first planar distal surface and the second planar distal surface.

24. The electronic assembly of claim 21, wherein the second space extends between a U-shaped portion of the holder.

25. The electronic assembly of claim 21, wherein:
the flexible circuit board further includes an intermediate section proximal to the distal section, wherein the intermediate section includes a center portion, a first side panel, and a second side panel, wherein the first side panel and the second side panel are angled relative to the center portion when the flexible circuit board is in the bent configuration.

26. The electronic assembly of claim 21, wherein the flexible circuit board further includes an intermediate section proximal to the distal section, the intermediate section including a central portion, a first lateral portion, and a second lateral portion on an opposite side of the central portion as the first lateral portion; wherein the second lateral portion includes an arm portion, and wherein the arm portion includes a surface configured to carry a camera of the first optical assembly.

27. The electronic assembly of claim 26, wherein, with the flexible circuit board in the unbent configuration, the central portion, the first lateral portion, and the second lateral portion are substantially coplanar, and
wherein, with the flexible circuit board in the bent configuration, the surface of the arm portion is substantially parallel to the distal section.

28. The electronic assembly of claim 27, wherein an optical axis of the first optical assembly extends perpendicular to and intersects the surface of the arm portion.

29. The electronic assembly of claim 23, wherein the first planar distal surface and the second planar distal surface are distal to an image sensor of the first optical assembly.

30. The electronic assembly of claim 21, wherein the holder extends distal to a fluid channeling component.

31. The electronic assembly of claim 21, wherein the space of the holder is configured to receive a portion of a fluid channeling component.

32. The electronic assembly of claim 21, wherein a first central longitudinal axis of the first optical assembly is transverse to a second central longitudinal axis of the second optical assembly.

33. An electronic assembly for a medical device, comprising:
a first optical assembly;
a second optical assembly;
a flexible circuit board having a plurality of bends, wherein the flexible circuit board comprises:
a front section configured to support the first optical assembly and including a U-shaped portion configured to receive the first optical assembly,
an intermediate section, and
a rear section, wherein a portion of each of the front, the intermediate, and the rear sections have a first wall thickness, wherein a first portion of the intermediate section has a second wall thickness, wherein a second portion of the rear section has a third wall thickness, wherein the first portion is coupled to the first optical assembly, and the second portion is coupled to the second optical assembly.

34. The electronic assembly of claim 33, further comprising a holder including:
a first planar distal surface at a distalmost end of the holder and extending transverse to a central longitudinal axis the first optical assembly;
a second planar distal surface at the distalmost end of the holder and extending transverse to the central longitudinal axis of the first optical assembly;
a first planar intermediate surface extending proximally from the first planar distal surface to a proximalmost end of the holder;
a second planar intermediate surface extending proximally from the second planar distal surface to the proximalmost end of the holder; and
a space extending 1) between the first planar distal surface and the second planar distal surface and 2) between the first planar intermediate surface and the second planar intermediate surface, wherein the space is configured to receive the optical assembly.

35. The electronic assembly of claim 33, wherein the first portion is proximal to the U-shaped portion.

36. The electronic assembly of claim 35, wherein the second portion is proximal to the first portion.

37. The electronic assembly of claim 33, wherein the intermediate portion includes an intermediate U-shaped portion configured to receive the second optical assembly.

38. An electronic assembly for a medical device, comprising:
a first optical assembly;
a second optical assembly;
a flexible circuit board having a bent configuration and an unbent configuration, and wherein the flexible circuit board comprises:
a distal section configured to hold the first optical assembly and including a first distal portion, a second distal portion, an opening configured to receive the first optical assembly, and a proximal portion coupled to the first and the second distal portions,
wherein a central longitudinal axis of the proximal portion extends between the first and the second distal portions when in the unbent configuration,
wherein, with the flexible circuit board in the unbent configuration, the proximal portion and the first and the second distal portions are substantially coplanar, and
wherein the first distal portion and the second distal portion are at a distalmost end of the flexible circuit board in the bent configuration and the unbent configuration of the flexible circuit board.

39. The electronic assembly of claim 38, further comprising a holder including:
a first planar distal surface extending transverse to a central longitudinal axis of the electronic assembly;
a second planar distal surface extending transverse to the central longitudinal axis of the electronic assembly; and
a space extending between the first planar distal surface and the second planar distal surface, wherein the space is configured to receive the first optical assembly.

40. The electronic assembly of claim 38, wherein:
the flexible circuit board further includes an intermediate section proximal to the distal section, wherein the intermediate section includes a center portion, a first side panel, and a second side panel, wherein the first side panel and the second side panel are angled relative to the center portion when the flexible circuit board is in the bent configuration; and
the first optical assembly includes an optical axis, wherein the optical axis extends in a plane substantially perpendicular to the distal section when the flexible circuit board in the bent configuration.
